(19) United States
(12) Patent Application Publication
(10) Pub. No.: US 2014/0170739 A1
(43) Pub. Date: Jun. 19, 2014

(54) INLINE SAMPLE FILTER FOR A FLOW CYTOMETER

(71) Applicant: Beckman Coulter, Inc., Brea, CA (US)

(72) Inventor: Richard A. Thomas, Miami, FL (US)

(73) Assignee: Beckman Coulter, Inc., Brea, CA (US)

(21) Appl. No.: 13/961,275

(22) Filed: Aug. 7, 2013

Related U.S. Application Data
(63) Continuation-in-part of application No. 13/696,277, filed on Jan. 22, 2013.
(60) Provisional application No. 61/693,771, filed on Aug. 27, 2012.
(60) Provisional application No. 61/680,645, filed on Aug. 7, 2012.
(60) Provisional application No. 61/331,795, filed on May 5, 2010.
(60) Provisional application No. 61/331,793, filed on May 5, 2010.
(60) Provisional application No. 61/331,789, filed on May 5, 2010.
(60) Provisional application No. 61/331,785, filed on May 5, 2010.

(51) Int. Cl. G01N 33/50

(52) U.S. Cl. CPC G01N 33/5005
USPC 435/288.7

(57) ABSTRACT

An inline sample filter for a flow cytometer.

### CROSS REFERENCE TO RELATED APPLICATIONS

This application claims priority to U.S. Ser. No. 61/693,771, titled INLINE SAMPLE FILTER FOR A FLOW CYTOMETER, filed Aug. 27, 2012, and to U.S. Ser. No. 61/680,645, titled SAMPLE FILTER FOR A FLOW CYTOMETER, filed Aug. 7, 2012. This application is also a Continuation-In-Part of U.S. Ser. No. 13/696,277, titled DIAGNOSTIC SYSTEM AND COMPONENTS, filed on Jan. 22, 2013, which claims priority to PCT/US2011/035420, titled DIAGNOSTIC SYSTEM AND COMPONENTS, filed on May 5, 2011, which claims priority to: U.S. Ser. No. 61/331,795, titled PROBE WASH STATION, filed on May 5, 2010; U.S. Ser. No. 61/331,793, titled EQUIPMENT INTERFACE, filed on May 5, 2010; U.S. Ser. No. 61/331,789, titled PROBE SENSING SYSTEM AND METHOD, filed on May 5, 2010; and U.S. Ser. No. 61/331,785, titled INFRARED FLUID DETECTION, filed on May 5, 2010. To the extent appropriate, a claim of priority is made to each of the above disclosed applications. Additionally, all of the above disclosed applications are hereby incorporated by reference in their entireties.

### BACKGROUND

In a flow cytometer, sample particles are passed through a small aperture in a flow cell (sometimes referred to as a measuring chamber). The small aperture confines the particles to a small known region where they can then be evaluated.

### SUMMARY

In general terms, this disclosure is directed to an inline filter for a flow cytometer.

One aspect is a flow cytometer comprising: a flow cell configured to pass sample particles through an aperture and past a detector to analyze the sample particles; and a fluid path connecting the flow cell to a sample container, the fluid path including: a sample inlet configured to receive sample particles in a sample fluid, a sample outlet configured to deliver the sample particles to the flow cell, a sample filter configured to retain particulate matter present in the sample fluid, a waste outlet configured to recover the particulate matter retained by the sample filter, and a junction fluidly connecting the sample inlet, the sample outlet, and the waste outlet, wherein the sample filter is disposed between the sample outlet and the junction.

Another aspect is a flow cytometer comprising: a flow cell configured to pass sample particles through an aperture, the aperture having a first diameter; an inline sample filter comprising: a filter tube including a first end and opposing second end, the filter tube including a fitting portion arranged at or adjacent to the second end; a filter plate arranged at the second end of the filter tube and including multiple filter apertures, wherein the filter apertures have a second diameter, and wherein the second diameter is equal to or less than the first diameter; and a first conduit coupled to the fitting portion of the filter tube and configured to deliver a sample to the inline sample filter; and a second conduit configured to deliver the sample, after filtering, to the flow cell.

A further aspect is an inline sample filter for use in a flow cytometer, the flow cytometer having a flow cell configured to pass sample particles through an aperture, the inline sample filter comprising: a filter tube including a first end and opposing second end, the filter tube including a fitting portion arranged at or adjacent to the second end; a filter plate arranged at the second end of the filter tube and including multiple filter apertures, wherein the filter apertures are sized to be equal to or smaller than the aperture but larger than the size of the particles to be passed through the aperture.

Another aspect is a method of filtering a sample using a sample filter.

Yet another aspect is a method of operating a flow cytometer.

### BRIEF DESCRIPTION OF THE DRAWINGS

FIG. 1 is a schematic block diagram of an example instrument including an inline filter and a protected aperture.

FIG. 2 is a schematic block diagram illustrating aspects of an example flow cytometer, including an example of instrument electronics.

FIG. 3 is a schematic block diagram illustrating additional aspects of the flow cytometer, including an example of a fluid transfer system.

FIG. 4 is a perspective view of an example inline filter.

FIG. 5 is a front view of the example inline filter shown in FIG. 4.

FIG. 6 is a cross-sectional side view of the example inline filter shown in FIG. 4.

FIG. 7 is a perspective side view of the inline filter shown in FIG. 4 connected to a filter tube.

FIG. 8 is a schematic side view of the inline filter and connected filter tube shown in FIG. 7 coupled to conduits of a fluid circuit of a flow cytometer.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

In certain instruments, fluid is passed through a fluid circuit including a small aperture. For example, in a flow cytometer a sample fluid is typically passed through a small aperture of a flow cell. The aperture is sized to reduce the number of particles that can pass through it at a time (i.e., to cause the particles to pass in single file), so that the content of the sample can be evaluated. A problem with many flow cytometers, however, is their propensity to clog. If the sample contains any particles, or aggregate of particles, greater in size than the cross-section of the aperture, the aperture may become clogged. The present disclosure describes an inline filter that reduces or eliminates the chance of clogging by filtering the sample. In some embodiments, the inline filter is positioned in the fluid circuit at a location just before the aperture, and operates to block the passage of particles that may otherwise clog the aperture. The aperture protected by the inline filter is sometimes referred to herein as a protected aperture.

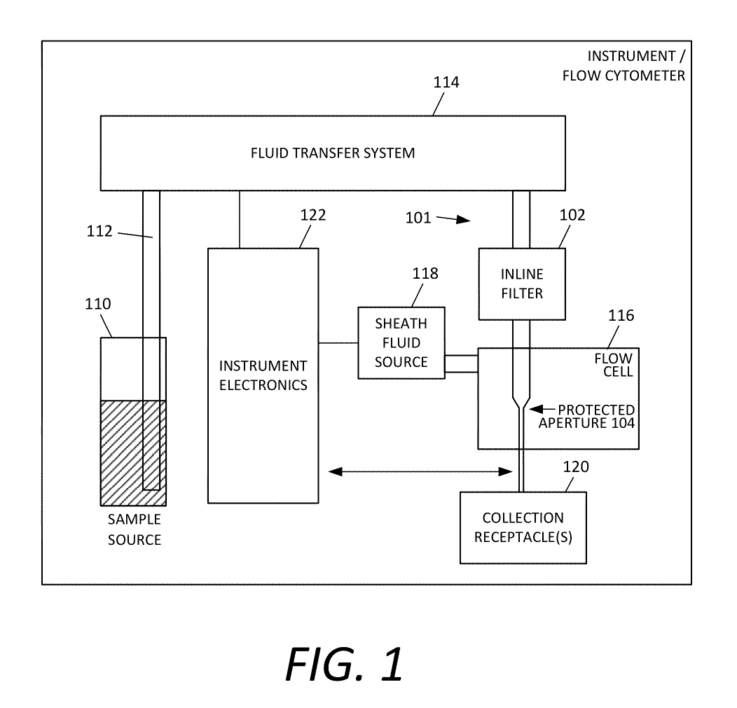

FIG. 1 is a schematic block diagram of an example instrument 100 including an inline filter 102 and a protected aperture 104. In this example, the instrument is a flow cytometer 100 including a sample source 110, a sample aspiration needle 112, a fluid transfer system 114, the inline filter 102, a flow cell 116 including the protected aperture 104, a sheath fluid source 118, a collection receptacle 120, and instrument electronics 122.

The instrument includes a fluid circuit 101 that delivers a fluid through the protected aperture 104. The protected aperture 104 has a reduced size that may become clogged by particles contained in the sample. An inline filter 102 is positioned upstream of the protected aperture 104 to block the passage of those particles, thereby protecting the protected aperture 104 from clogging.

In the example depicted in FIG. 1, the flow cytometer includes a sample source 110. An example of a sample source 110 is a test tube containing a sample. Other receptacles or other sample sources are used in other embodiments.

A sample aspiration needle 112 is provided in some embodiments to extend into the sample source 110 for receiving the sample from the sample source 110. The sample aspiration needle includes one or more apertures therein through which the sample can be received from the sample source.

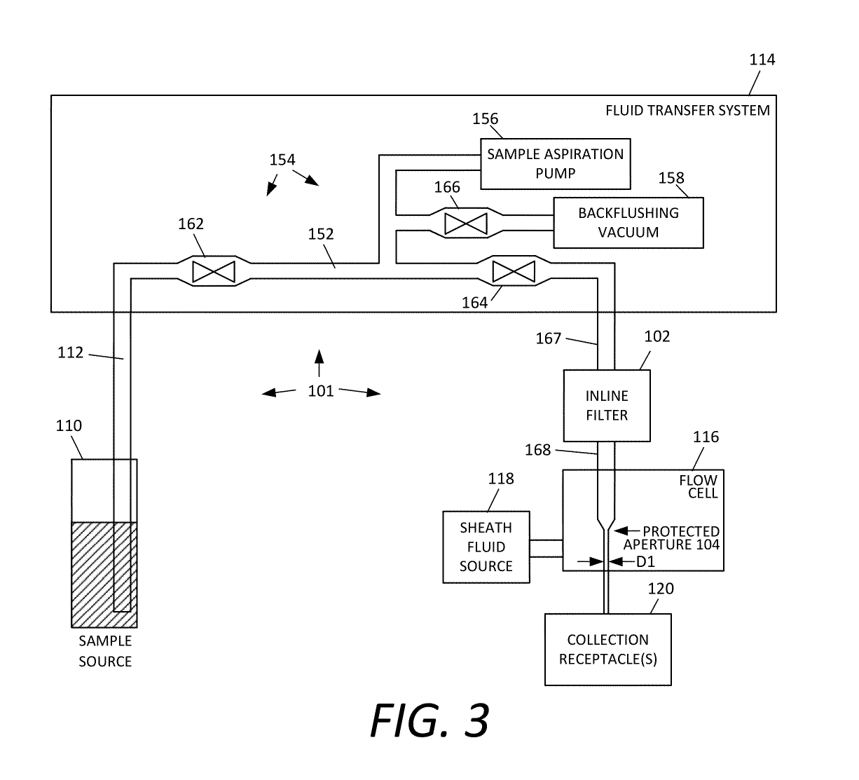

A fluid transfer system 114 is arranged and configured to deliver the sample along the fluid circuit 101 from the sample source 110 to the flow cell 116. A more detailed example of a fluid transfer system 114 is illustrated and described herein with reference to FIG. 3.

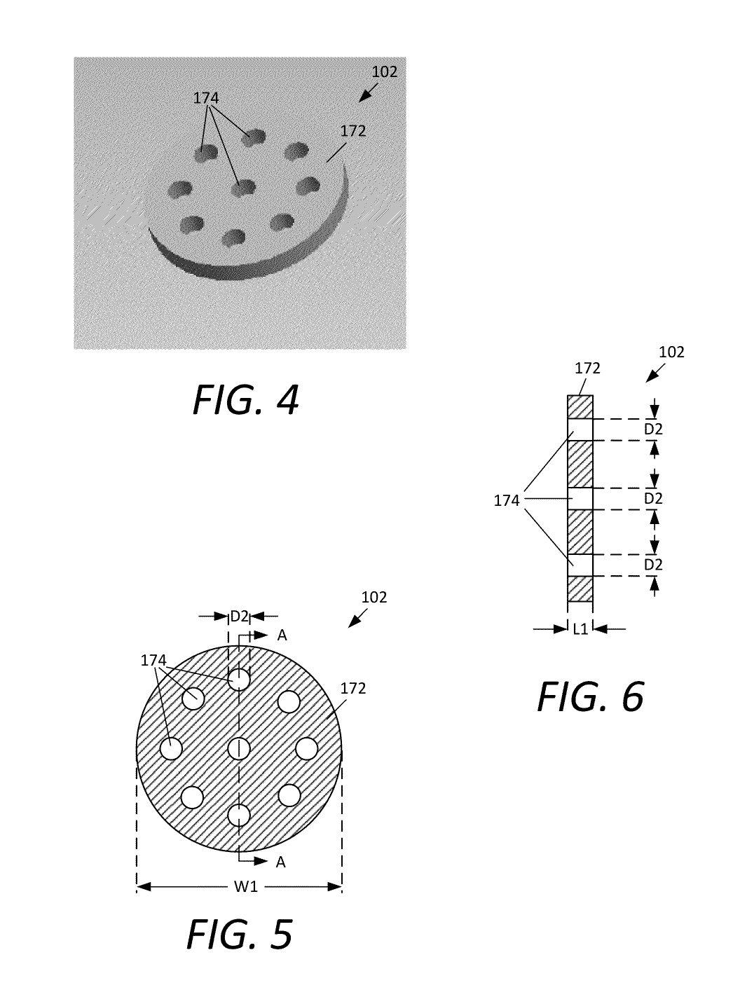

As the sample passes along the fluid circuit 101, it passes through the inline filter 102. The inline filter 102 blocks the passage of particles so that the particles do not pass downstream of the inline filter 102. This operates to protect the protected aperture 104 of the flow cell 116 to prevent or reduce clogging of the protected aperture 104. The inline filter 102 is arranged and configured so that even after the inline filter has blocked one or more particles from the sample, the rest of the particles can continue unimpeded along the fluid circuit and into the flow cell 116 and protected aperture 104. An example of inline filter 102 is illustrated and described in more detail with reference to FIGS. 4-6.

The flow cell 116 (sometimes referred to as a measuring chamber) includes the protected aperture 104 which is protected from clogging by the inline filter 102. The fluid circuit 101 becomes narrower at the protected aperture 104, to attempt to reduce the number of particles passing at once through the aperture 104 so that each particle can be evaluated.

A sheath fluid source 118 is provided in some embodiments. As noted above, the sheath fluid source 118 supplies a sheath fluid to the flow cell 116 where it is mixed with the sample. As discussed in more detail herein, the sheath fluid source is also used in some embodiments as a backflushing fluid to clean the fluid circuit 101 and remove trapped particles from the inline filter 102.

In some embodiments, the fluid circuit 101 terminates at one or more collection receptacles 120 where the sample is collected and stored for subsequent use or disposal.

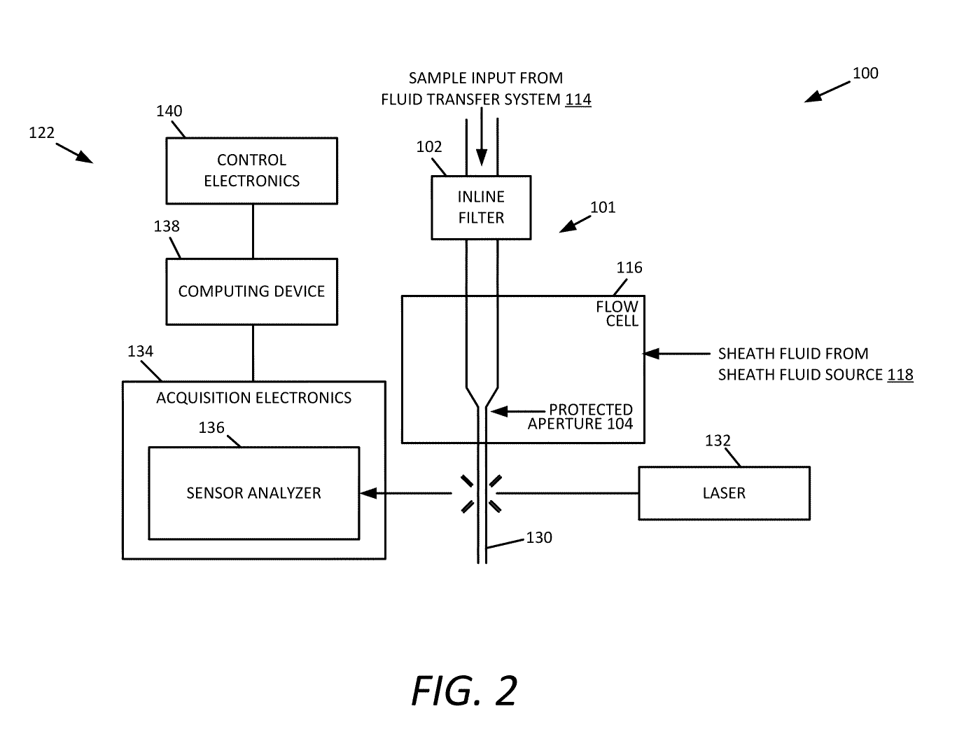

The instrument electronics 122 operate to control the operation of the flow cytometer and to analyze the content of the sample. An example of the instrument electronics 122 is illustrated and described in more detail with reference to FIG. 2.

FIG. 2 is a schematic block diagram illustrating additional aspects of an exemplary flow cytometer 100, including an example of the instrument electronics 122.

In this example, the flow cytometer 100 includes an inline filter 102, a flow cell 116 including a protected aperture 104, and the instrument electronics 122. The example instrument electronics 122 include a laser 132, acquisition electronics 134 including a sensor analyzer 136, a computing device 138, and control electronics 140.

The fluid circuit 101 receives sample from the sample source (shown in FIG. 1) and provides the sample to the protected aperture 104, which in this example is within the flow cell 116. An inline filter 102 is arranged upstream of the protected aperture to prevent the passage of particles that may otherwise clog the protected aperture 104.

The sample is then analyzed by the instrument electronics 122, such as by illuminating the sample stream 130 from the flow cell 116 with a laser beam from laser 132. Acquisition electronics 134, such as including a sensor analyzer 136, detect characteristics of the sample, such as the way that the laser beam is scattered by the particles.

A computing device 138 receives signals and/or data from the acquisition electronics 134 and interacts with the user to display data relating to the characteristics of the particles in the sample.

Control electronics 140 are also included in some embodiments that interact with the computing device to control the operation of the flow cytometer 100.

The principles described herein can be implemented in various types of flow cytometers 100 in various possible embodiments. For example, some embodiments involve a sorting flow cytometer, while other embodiments involve a non-sorting flow cytometer. When implemented as a sorting flow cytometer, the flow cytometer 100 typically includes sorting control electronics as part of the control electronics 140, a vibration generator coupled to the fluid nozzle (which may be part of or arranged after the flow cell, for example), and sorting plates electrically coupled to electrical charge generators, which generate an electric field therebetween to direct drops as they separate from the sample stream 130 into appropriate collection receptacles 120 (shown in FIG. 1).

FIG. 3 is a schematic block diagram illustrating additional aspects of an exemplary flow cytometer 100, such shown in FIG. 1, including an example of the fluid transfer system 114.

As shown in FIG. 1, the flow cytometer includes the sample source 110, the fluid transfer system 114, the inline filter 102, the flow cell 116 including the protected aperture 104, the sheath fluid source 118, and the collection receptacle(s) 120. In the example shown in FIG. 3, the fluid transfer system 114 includes conduits 152, valves 154, a sample aspiration pump 156, and a backflushing vacuum 158. The valves 154 include valves 162, 164, and 166. The valves are selectively opened and closed by control electronics 140, shown in FIG. 2, for example. In some embodiments, a valve 154 may also be provided before the sample aspiration pump 156 to selectively open or close the conduit leading to the sample aspiration pump 156.

During the analysis of a sample, the sample is directed from the sample source 110 through the fluid circuit 101. In this example, the fluid circuit 101 passes the sample through the sample aspiration needle 112, through the conduits 152 and valves 154 of the fluid transfer system 114, through the inline filter 102 and the flow cell 116, and into the collection receptacle(s). The flow cell 116 includes the protected aperture 104, through which the sample is passed. A cross-sectional distance across the protected aperture is represented in FIG. 3 by distance D1. In some embodiments, the cross-sectional distance D1 is a maximum distance.

The protected aperture 104 can have various cross-sectional shapes, such as a circular, rectangular, or triangular shape. The cross-sectional distance D1 is typically in a range from about 50 microns to about 500 microns.

In some embodiments, a sample aspiration pump 156 operates to retrieve the sample from the sample source 110 and transfer the sample to the flow cell 116. To begin, the valve 162 is opened and valves 164 and 166 are closed. The sample aspiration pump then retrieves a volume of the sample from the sample source 110 through the conduits 152 and valve 162 by reducing the pressure in the conduit 152. In some embodiments, a portion of the conduit forms a sample loop, which has a suitable volume for temporarily storing the volume of the sample retrieved from the sample source 110.

Once the desired volume of the sample has been retrieved, the valve 162 is closed and valve 164 is opened. The sample aspiration pump 156 is then reversed to increase the pressure in the conduit 152, thereby causing the sample to flow through valve 164, through the inline filter 102, through the flow cell, and into the collection receptacle(s) 120.

After a sample has been evaluated, the fluid circuit 101 can be cleansed through a backflushing operation. The backflushing operation also operates to remove any particles that may have been blocked by the inline filter 102. The backflushing operation is performed by closing the valve 162, keeping valve 164 open, and opening valve 166. The backflushing vacuum 158 is then turned on, causing a suction to be applied to the conduit 152. The suction draws sheath fluid from the sheath fluid source 118 in the flow cell 116 up through the inline filter and through valves 164 and 166. The sheath fluid and any remaining particles are can then be directed to a waste receptacle, such as one of the collection receptacles 120, or another receptacle. The backflushing operation draws the sheath fluid through the inline filter 102 at a sufficient velocity that any particles trapped in the inline filter are dislodged from the inline filter 102. The backflushing velocity during the backflushing operation is typically much larger than the velocity at which the sample is passed through the inline filter 102 during normal operation. In some embodiments, one or more additional cleansing operations can similarly be performed to clean additional portions of the fluid circuit 101.

In some embodiments, the inline filter 102 is connected to conduits 167 and 168. The conduit 167 is connected upstream of the inline filter 102, such as to provide a fluid path between the fluid transfer system 114 and the inline filter 102. The conduit 168 is connected downstream of the filter, such as to provide a fluid path between the inline filter 102 and the flow cell 116. An example of a conduit 167,168 is silicon tubing.

One advantage of arranging the inline filter 102 just upstream of the flow cell 116 is that the fluid velocity at this point is relatively low, which reduces the chance of shearing or otherwise damaging particles as they interact with the inline filter 102. As one example, the fluid velocity is on the order of magnitude of 10 to 100 micro liters per minute.

FIGS. 4-6 illustrate an example of an inline filter 102.

FIG. 4 is a perspective view of the inline filter 102. In this example, the inline filter 102 includes a body 172 including apertures 174 formed therein.

In some embodiments, the inline filter 102 exhibits one or more of the following characteristics: (1) it permits gentle movement of certain desired particles (such as cells) through the filter without damaging the cell walls, (2) it blocks particles having a size that would otherwise clog the protected aperture; (3) it can be arranged within the flow cytometer at a location that allows complete cleaning of the filter between samples, and (4) it permits fluid to pass through the filter at a low velocity to reduce jamming of particles into the filter apertures.

The body 172 of the inline filter 102 is typically formed of a piece of material, such as a sheet of stainless steel metal. Other materials can be used, provided that such materials do not significantly corrode or otherwise deteriorate when exposed to the materials that are passed through the fluid circuit 101. Other examples of possible materials are glass and plastic.

The apertures 174 are sized small enough to block particles from passing through that are likely to clog the protected aperture 104, shown in FIGS. 1-3, but are sized large enough that they do not block the particles of interest.

The apertures 174 can have any desired shape. In this example the apertures have a circular cross-sectional shape. A benefit of a circular cross-sectional shape is that it has a substantially constant cross-sectional distance. Another benefit of a circular cross-sectional shape is that it reduces sharp corners, which could otherwise damage particles passing therethrough. However, other embodiments include apertures 174 having other shapes, such as triangular, square, rectangular, pentagonal, or different shapes. In some embodiments, the walls forming edges of the apertures 174 are smooth.

Two or more apertures 174 are provided so that even when one of the apertures 174 becomes blocked by an undesired particle, one or more of the other apertures 174 remain open to permit continued flow of the sample. In this example, the inline filter 102 includes nine apertures. Other embodiments have other quantities of apertures. Typically a larger quantity of apertures is preferred, limited by the size of the body 172 and the precision of the aperture forming techniques, for example. An advantage of having a larger quantity of apertures is that a greater quantity of particles can be trapped by the filter without clogging the filter 102. Another advantage of having a larger quantity of apertures is that it reduces the velocity of fluid flow through the apertures. A lower velocity is preferred to reduce shearing of delicate particles, such as cell walls.

FIG. 5 is a front view of the example inline filter 102. As described above, the example inline filter 102 includes a body 172 and apertures 174.

In this example, the body 172 has a circular cross-sectional shape having a width W1 (which is consequently also the height and the diameter). Other embodiments have other cross-sectional shapes, as desired. The body 172 can have various possible shapes and sizes. In some embodiments, the body 172 has a width W1 in a range from about 0.01 inches to about 0.5 inches. In another example embodiment, the body 172 has a width W1 of about 0.062 inches.

The apertures 174 extend through the body 172. In some embodiments, the apertures 174 are formed in the body by an aperture forming process. An example of an aperture forming process is drilling. Another example of an aperture forming process involves photolithography.

In some embodiments, the apertures 174 have a cross-sectional distance D2. In the illustrated example, the apertures 174 have a circular cross-section, such that the distance D2 is also the diameter. However, other embodiments can have other cross-sectional shapes. In some embodiments, the cross-sectional distance D2 is a maximum cross-sectional distance.

The cross-sectional distance D2 is selected to block particles that may clog the protected aperture 104, while permitting other smaller particles to pass through. In particular, the cross-sectional distance D2 of the protected aperture 104 should not be less than (or at least not significantly less than) the cross-sectional distance of particles that are to be analyzed by the flow cytometer 100.

Several exemplary dimensions will now be described, but other embodiments can have other dimensions. As one example, a flow cytometer 100 has a protected aperture with a cross-sectional distance D1 (shown in FIG. 3) of 180 microns, and is utilized to analyze particles having a 10 micron cross-section. In this example, the inline filter 102 is configured to have apertures 174 that are sized smaller than or equal to the cross-sectional distance D1, but also to have apertures 174 that are sized larger than the cross-section of the particle. For example, the cross-sectional distance D2 is in a range from about 50 microns to about 180 microns, or in a range from about 100 microns to about 170 microns, and preferably about 150 microns.

In some embodiments, the apertures 174 are selected to have a cross-sectional distance D2 that is less than 5 times the maximum cross-sectional distance D1 of the protected aperture 104 (FIG. 3). Further, in some embodiments the apertures 174 are selected to have a cross-sectional distance D2 that is greater than or equal to 2 times the cross-section of the particles of interest.

FIG. 6 is a cross-sectional side view of the example inline filter 102 taken along cross-section A-A shown in FIG. 5. The inline filter 102 includes body 172 and apertures 174.

The cross-sectional distance D2 of several of the apertures 174, which is discussed in more detail herein with reference to FIG. 5, is also visible in FIG. 6.

Additionally, FIG. 6 illustrates a thickness of the body 172, which is also the length L1 of apertures 174. In some embodiments, the length L1 is less than 50 times the cross-sectional distance D2 of the apertures 174. As one example, the body 172 is made of 125 micron 316 stainless steel stock, such that the length L1 of apertures 174 are about 125 microns. Other embodiments have other lengths. Shorter lengths L1 are beneficial in reducing the interaction between the walls of the apertures 174 and the particles, which may otherwise damage certain particles (such as cell walls).

In some embodiments, the inline filter 102 has apertures 174 with a low length (L1) to cross-sectional distance (D2) ratio. In some embodiments, the apertures 174 have a length (L1) to cross-sectional distance (D2) ratio of about 0.8.

In some embodiments, the apertures 174 are tapered so that the apertures 174 are wider in the upstream direction than in the downstream direction.

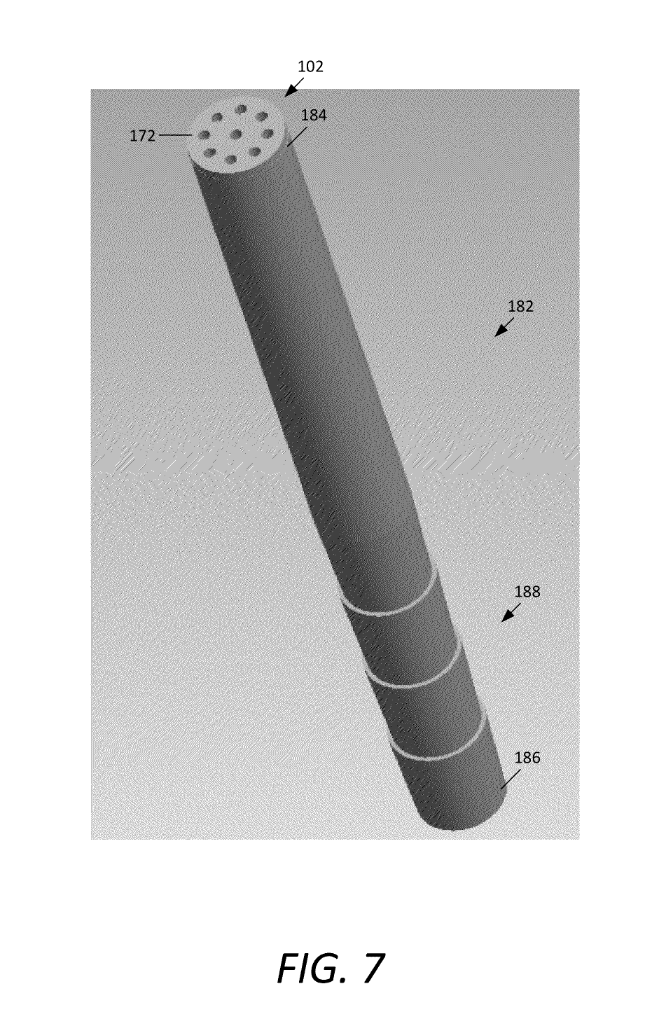

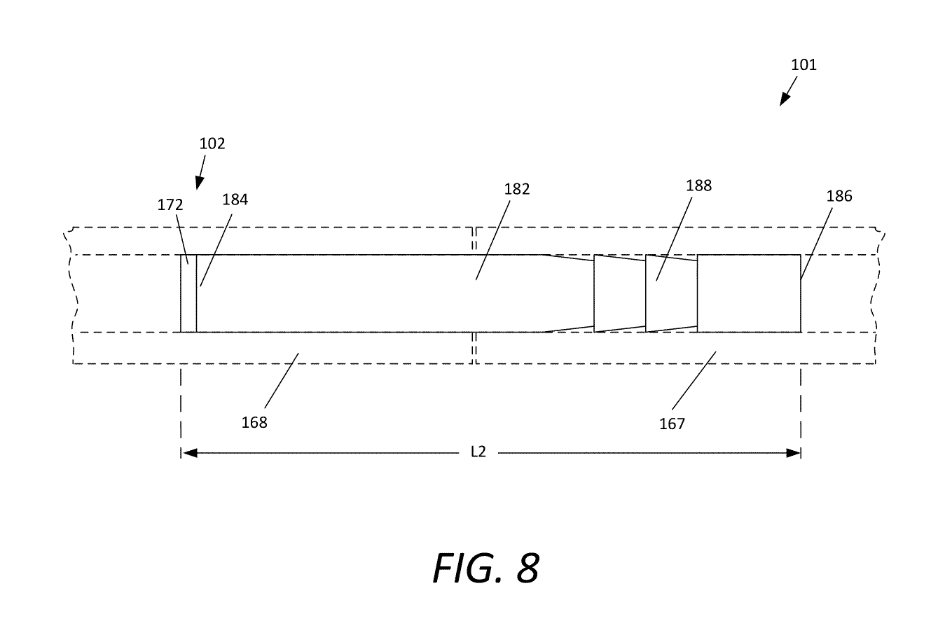

FIGS. 7 and 8 illustrate another example of the inline filter 102, in which the inline filter 102 is connected to a filter tube 182. The filter tube 182 supports the inline filter 102 in an appropriate location in the fluid circuit 101 of the flow cytometer 100.

FIG. 7 is a perspective view of the inline filter 102 and filter tube 182.

The filter tube 182 is formed of a tube of material. In one example embodiment, the tube is formed of ⅙″ 316 stainless steel tubing. Other materials are used in other embodiments, such as glass or plastic.

In this example, the filter tube 182 includes opposing first and second ends 184 and 186. The inline filter 102 is connected to the first end 184. In some embodiments, the inline filter 102 is welded to the first end 184 of the filter tube 182. In some embodiments, the inline filter 102 includes welding tabs that extend out from edges of the inline filter. The welding tabs can be bent down toward the filter tube to assist with formation of a solid weld joint. Other fastening techniques can alternatively be used to connect the inline filter 102 with the end 184 of the filter tube 182.

Adjacent the second end is a fitting portion 188. The fitting portion 188 has a multi-tiered construction that is widest at the end of each tier closest to the first end 184, and gradually tapers inward toward the second end 186. Ridges are formed at the widest end of each tier. The fitting portion 188 assists the user in inserting the inline filter 102 and the filter tube 182 into the fluid circuit 101, as shown in FIG. 8.

FIG. 8 is a schematic side view of the inline filter 102 and the filter tube 182 inserted into a portion of the fluid circuit 101.

In some embodiments, the inline filter 102 and filter tube 182 are coupled to conduits 167 and 168, as shown in FIG. 3. The first conduit 167 couples the inline filter 102 with the fluid transfer system, while the second conduit 168 couples the inline filter 102 with the flow cell 116.

One exemplary process for inserting the inline filter 102 into the fluid circuit 101 is as follows. The second end 186 of the filter tube 182 is first inserted partially into the free end of the first conduit 167. The fitting portion 188 has a tiered construction with tapered tiers that are oriented so that they do not oppose the insertion of the filter tube 182 into the first conduit 167.

The first conduit 167 is then held and gently squeezed while inserting the inline filter 102 and first end 184 of the filter tube 182 into the free end of the second conduit 168. When the conduit 167 is squeezed at the fitting portion 188 of the filter tube 182, the fitting portion 188 helps to prevent further movement of the filter tube 182 into the first conduit 167, while allowing adequate force to be applied to insert the inline filter 102 and first end 184 of the filter tube 182 into the conduit 168.

The inline filter 102 and filter tube 182 have a combined length L2. In some embodiments, the length L2 is in a range from about 300 thou to about 400 thou. Other embodiments have longer or shorter lengths.

In another possible embodiment, the inline filter 102 is connected to another part of the flow cytometer 100. As one example, the inline filter 102 is connected directly to an upstream end of the flow cell 116. More specifically, the inline filter 102 can be connected to the upstream end of the sample injector needle. In this example, the inline filter 102 is not a separate component, but rather is physically connected as part of the sample injector needle.

In yet other possible embodiments, the inline filter 102 can be arranged anywhere along the fluid circuit 101 upstream of the protected aperture 104.

In addition to the use of the inline filter 102 in a flow cytometer 100 as primarily described herein, the inline filter 102 can similarly be used to prevent clogging of any small aperture utilizing the same principles disclosed herein. For example, the inline filter 102 can be used in a fluidic instrument, such as with micro-channel plates, or other instruments.

The terms upstream and downstream are sometimes used herein. Downstream refers to the direction that the sample flows through the fluid circuit 101 starting at the sample source 110 and ending at the collection receptacles. Upstream refers to the direction opposite the downstream direction. It is recognized that fluid flow may not always be in this direction, such as during a backflushing operation.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A flow cytometer comprising:
a) a flow cell configured to pass sample particles through an aperture and past a detector to analyze the sample particles; and
b) a fluid path connecting the flow cell to a sample container, the fluid path including:
i. a sample inlet configured to receive sample particles in a sample fluid,
ii. a sample outlet configured to deliver the sample particles to the flow cell,
iii. a sample filter configured to retain particulate matter present in the sample fluid,
iv. a waste outlet configured to recover the particulate matter retained by the sample filter, and
v. a junction fluidly connecting the sample inlet, the sample outlet, and the waste outlet,
wherein the sample filter is disposed between the sample outlet and the junction.

2. A flow cytometer comprising:
a flow cell configured to pass sample particles through an aperture, the aperture having a first diameter;
an inline sample filter comprising:
a filter tube including a first end and opposing second end, the filter tube including a fitting portion arranged at or adjacent to the second end;
a filter plate arranged at the second end of the filter tube and including multiple filter apertures, wherein the filter apertures have a second diameter, and wherein the second diameter is equal to or less than the first diameter; and
a first conduit coupled to the fitting portion of the filter tube and configured to deliver a sample to the inline sample filter; and
a second conduit configured to deliver the sample, after filtering, to the flow cell.

3. An inline sample filter for use in a flow cytometer, the flow cytometer having a flow cell configured to pass sample particles through an aperture, the inline sample filter comprising:
a filter tube including a first end and opposing second end, the filter tube including a fitting portion arranged at or adjacent to the second end; and
a filter plate arranged at the second end of the filter tube and including multiple filter apertures, wherein the filter apertures are sized to be equal to or smaller than the aperture and larger than the size of the particles to be passed through the aperture.

4. A flow cytometer comprising:
a fluid transfer system configured to retrieve a sample from a sample container and provide the sample along a fluid path;
a flow cell in fluid communication with the fluid transfer system; and
an inline filter arranged upstream of the flow cell to filter the sample before the sample is introduced into the flow cell.
